(19) United States
(12) Patent Application Publication
(10) Pub. No.: US 2022/0208308 A1
(43) Pub. Date: Jun. 30, 2022

(54) COMPUTER SYSTEM AND METHOD THEREOF

(71) Applicants: Hitachi, Ltd., Tokyo (JP); KYOTO UNIVERSITY, Kyoto (JP)

(72) Inventors: Kiyoto ITO, Tokyo (JP); Shiori NAKAZAWA, Tokyo (JP); Osamu IMAICHI, Tokyo (JP); Michihiro ARAKI, Kyoto (JP)

(21) Appl. No.: 17/552,861

(22) Filed: Dec. 16, 2021

(30) Foreign Application Priority Data
Dec. 28, 2020 (JP) 2020-218498

(51) Int. Cl. G16B 50/30; G16B 45/00; G16B 40/20

(52) U.S. Cl. CPC G16B 50/30; G16B 40/20; G16B 45/00

(57) ABSTRACT

A computer system that supports design for improving a function of a biological resource registers a history of the design, the history of the design including pair information of a related element related to a property of the biological resource and an operation on the related element, searches a database based on the pair information, acquires additional information other than the related element, the additional information being information related to the property of the biological resource based on a result of the search, computes a correlation of the additional information to the related element, and evaluates the additional information based on the calculated correlation.

### CROSS-REFERENCE TO RELATED APPLICATION

The present application claims priority from Japanese application JP2020-218498 filed on Dec. 28, 2020, the contents of which is hereby incorporated by reference into this application.

### BACKGROUND OF THE INVENTION

### 1. Field of the Invention

The present invention relates to a computer system, and more particularly to a computer system that supports design for artificial improvement and improvement of biological functions such as high-productivity microorganism generation.

### 2. Description of the Related Art

In recent years, with the remarkable progress of biotechnology such as genetic modification and genome editing, synthetic biotechnology has attracted attention, for example, creation of a microorganism by artificially enhancing a substance production ability possessed by an organism by a technique such as genetic modification. In the development of such a highly productive microorganism, in order to express a desired biological function in a cell, a gene modification technique such as newly introducing a gene not originally possessed by the microorganism or enhancing, deleting, or suppressing the expression of a native gene in the cell is required.

Conventionally, as one of them, it has been attempted to improve microorganisms by executing a metabolic simulation by applying a flux balance analysis based on a metabolic map of a certain organism and predicting a metabolic flux flowing in the metabolic map (for example, JP 2005-58226 A).

On the other hand, a search device has been proposed in which, in order to search for a bioitem (gene) related to improvement of a biological function, a bioitem document set is stored for each bioitem, in each bioitem document set, a keyword is searched from the bioitem document set, the number of documents Nh including the keyword in the bioitem document set is acquired for each bioitem, a bioitem of which the number of documents Nh is 1 or more is selected as a candidate bioitem, a document count table including a) the number of documents Nh and/or b) the number of documents not including the keyword and including a bioitem name is created for each candidate bioitem, a correlation score between the bioitem and the keyword is calculated based on statistical calculation using the document count table, and a candidate bioitem is output based on the calculated correlation score (WO 2007/126088).

### SUMMARY OF THE INVENTION

In a metabolic simulation based on flux balance analysis, not only genes not included in a metabolic model are not targeted, but also a solution is obtained by a multidimensional equation, so that the solution may be indefinite or may not be analyzable, and thus, the metabolic simulation is insufficient as an assistance system for developing a high-productivity microorganism.

On the other hand, in a search device that calculates a correlation score between a bioitem and a keyword based on a keyword search and the number of documents hit by the search and outputs a candidate bioitem, there is a problem that when a biological function of a high-productivity microorganism is focused on for the development of the high-productivity microorganism, a large number of related genes are hit and effective genes cannot be narrowed down, and when a specific gene is focused on, effectiveness for a desired biological function cannot be searched.

Therefore, a computer system useful as an assistance system for developing a high-productivity microorganism has not yet been realized, but in reality, genetic modification of a high-productivity microorganism is not completely freed from the accumulation of trial and error supported by the knowledge and experience of an individual researcher, and waste of research resources such as researchers and facilities, research funds, and the like cannot be avoided.

Therefore, an object of the present invention is to provide a computer system suitable for efficiently performing design support for improving the properties of biological resources such as highly productive microorganisms, and thereby developing biological resources useful for human beings while avoiding waste of research resources, research funds, and the like.

In order to achieve the above object, the present invention provides a computer system that supports design for improving a function of a biological resource. The computer system includes a controller that executes a program recorded in a memory. The controller is configured to: register a history of the design, wherein the history of the design includes pair information of a related element related to a property of the biological resource and an operation on the related element; search a database based on the pair information; acquire, based on a result of the search, additional information other than the related element, the additional information being information related to a property of the biological resource; compute a correlation of the additional information to the related element; and evaluate the additional information based on the computed correlation.

According to the present invention, it is possible to provide a computer system suitable for efficiently performing design support for improving the properties of biological resources such as highly productive microorganisms, and thereby developing biological resources useful for human beings while avoiding waste of research resources, research funds, and the like.

### BRIEF DESCRIPTION OF THE DRAWINGS

FIG. 1 is a block diagram of an embodiment of a computer system according to the present invention;

FIG. 2 illustrates an example of a data structure in a design history database of a computer system in FIG. 1;

FIG. 3 is an example of a management table for managing a data group of the design history database;

FIG. 4 is an example of a user interface in a user computer of the computer system;

FIG. 5 is a flowchart illustrating an operation of a server computer of the computer system;

FIG. 6 is a block diagram illustrating the principle of search of candidate genes;

FIG. 7 is an example of a management table of candidate genes;

FIG. 8 is an example of a ranking table of candidate genes;

FIG. 9 is a block diagram illustrating an example of a relationship among a metabolic map, a target gene, and a candidate gene; and

FIG. 10 is a block diagram of a computer system according to a second embodiment.

### DESCRIPTION OF THE PREFERRED EMBODIMENTS

Next, embodiments of the invention will be described. This embodiment describes a computer system that supports improvement of a function of a high-productivity microorganism (artificial microorganism) as a biological resource. Specifically, the computer system proposes, to a user, a useful gene that can be a candidate for improving the function of the artificial microorganism as a candidate gene by analyzing a history (past information) of genetic modification including introduction of a new gene into the high-productivity microorganism, enhancement of a function of a conventional gene, disruption of a conventional gene, suppression of a conventional gene, and the like, to produce a substance useful for the high-productivity microorganism, improve production efficiency of the useful substance, and the like.

With reference to the candidate gene proposed by the system, the user can verify whether there is an effect in improving the function, activity, performance, property, characteristic, attribute, or the like of the artificial microorganism by applying wet manipulation to the artificial microorganism. For the user, the burden for searching for candidate genes is greatly reduced.

The biological resources are those having some biological activity such as animal and plant cells, microorganisms, bacteria, viruses, hormones, enzymes, tissues, organs, viscera, genes, chromosomes, DNA, RNA, and artificial microorganisms (high-productivity microorganisms). The biological resource may be referred to as a biological material or a biological ingredient. Genes are fundamental as elements related to functional modification of organisms, cells, and microorganisms. Metabolism of an artificial microorganism is modeled as a metabolic map, and genetic modification leads to establishment, blocking, suppression, activation, and the like of a metabolic pathway in the metabolic map. By modifying genes, metabolic functions can be modified on a large scale, and as a result, for example, useful substances that can be produced by microbial fermentation can be diversified, and the yield thereof can be improved as much as possible. “Modifying Genes” includes introducing novel genes, repressing native genes, enhancing native genes, and disrupting native genes.

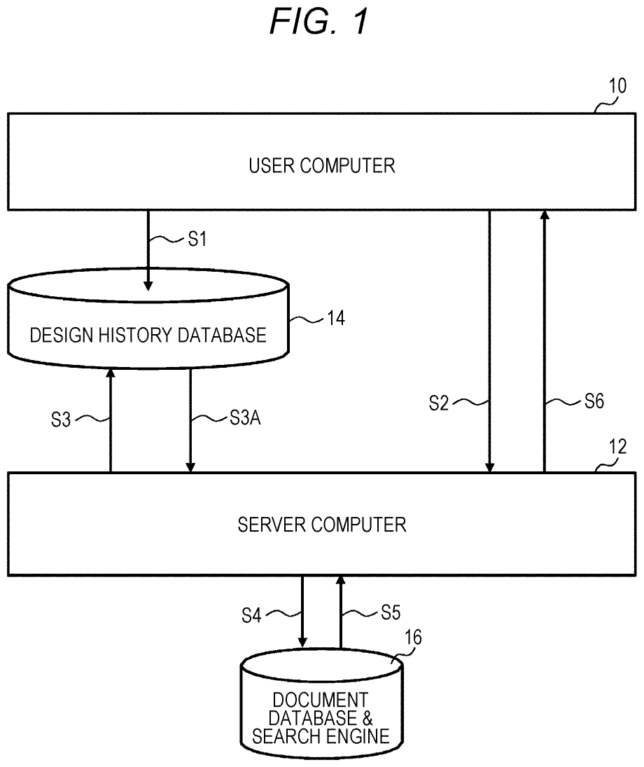

FIG. 1 is an example of a block diagram of a computer system. The computer system includes a user computer 10, a server computer 12, and a communication network (Internet, LAN, etc.) that connects the user computer and the server computer. Each of the user computer 10 and the server computer 12 includes a controller (CPU: control unit), a memory, and an input/output interface, which are normal hardware resources. A plurality of databases are connected to the communication network, and there are a design history database 14 and a document (related information) database & search engine 16. Note that the computer system may be configured by a personal computer.

The design history database 14 may exist for each item such as an organization, an association, a country, an institution, a region, or a period. The design history database 14 may be singular or plural. The same applies to the document database & search engine 16.

The design history is a history of metabolic design of a biological resource, and includes a plurality of pieces of pair information of a gene (related element) and an operation for the gene in the biological resource. The operation includes an operation for modifying a gene in order to improve a property, characteristic, performance, function, or attribute of a biological resource, and specific examples thereof include introduction of a new gene, enhancement of an existing gene, suppression thereof, and disruption thereof to a high-productivity microorganism.

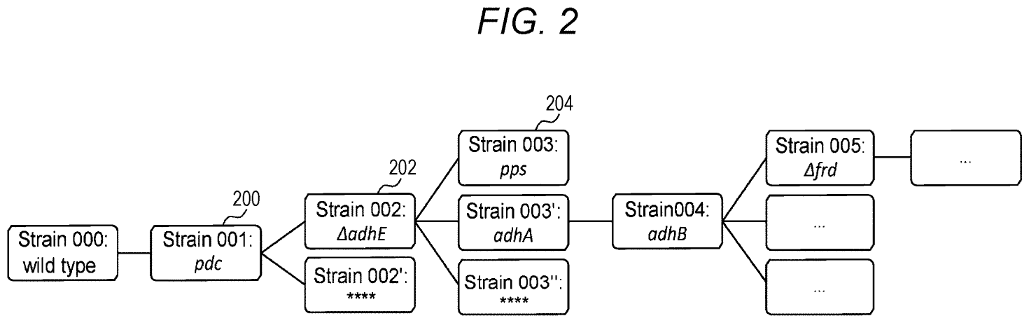

FIG. 2 illustrates an example of a data structure in the design history database 14. This data structure is described as a tree structure in which a plurality of modification lists are connected by links. One block in FIG. 2 corresponds to the modification list. As illustrated in FIG. 2, the construction of the artificial microorganism is realized by stacking genetic modifications starting from a predetermined parent strain. One block (modification list) includes, for each modification generation, pair information of a gene name to be manipulated and manipulation for the gene. The plurality of blocks are linked according to the generation of modification.

Reference numeral 200 denotes a modification list in which the gene pdc is introduced from the Wild type (strain 000 parent strain) in the modified strain 1 (strain 001), reference numeral 202 denotes a modification list in which the gene adhE is disrupted (A) from the modified strain 1 (strain 001) in the modified strain 2 (strain 002), and reference numeral 204 denotes a modification list in which the gene pps is added to the modified strain 2 (strain 002) in the modified strain 3 (strain 003). Therefore, the reference numeral 204 indicates that the parent strain is a modified strain in which adhE is disrupted and pdc and pps are added.

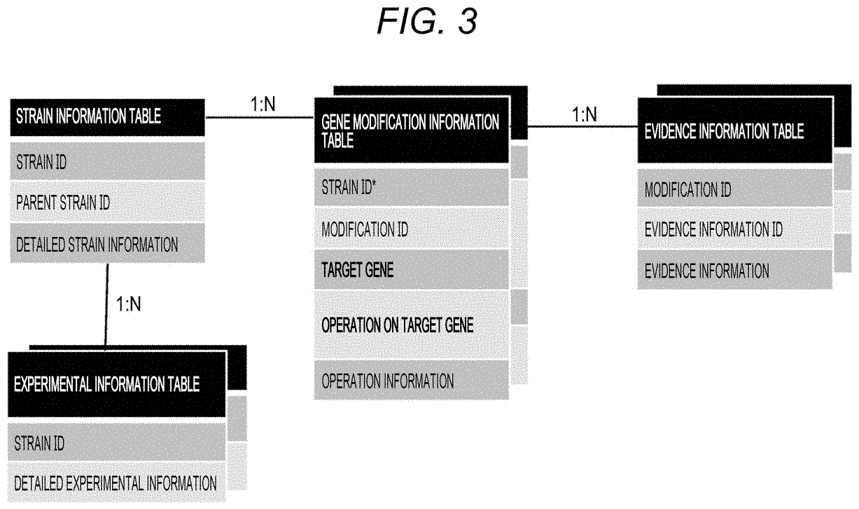

FIG. 3 illustrates an example of a management table for managing a data group of the design history database 14. The management table includes a strain information table for managing strains of microorganisms, a gene modification information table for managing modification information of genes of strains, an evidence information table for managing evidence of modification, and an experimental information table. The strain information table includes a strain ID, a parent strain ID, and detailed strain information. The gene modification information table includes a strain ID, a modification ID, a target gene name, an operation name for the target gene, and operation information, and has a 1:N correspondence with the strain information table using the strain ID as a key.

The evidence information table includes a modification ID, an evidence information ID, and evidence information, and has a 1:N correspondence with the evidence information table using the modification ID as a key. The experimental information table includes a strain ID and detailed experimental information, and has a 1:N correspondence with the strain information table using the strain ID as a key.

The document database & search engine 16 accumulates data groups related to biological resources, and the attribute of data is not limited to text data, image data, audio data, and the like, and the type of data is mainly document data such as papers, patents, books, and journals, but is not limited thereto.

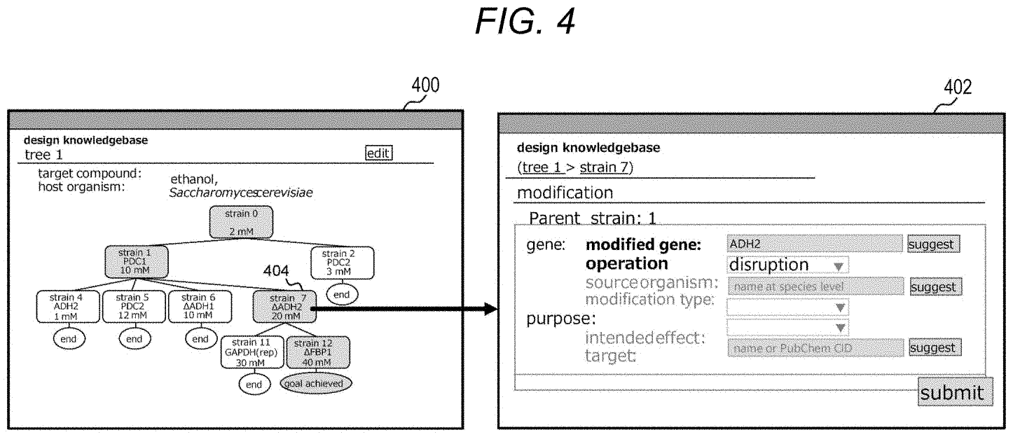

The user computer 10 includes an interface for inputting (S1) a modification list as a design history to the design history database 14. FIG. 4 is an example of a graphic user interface, and the graphic user interface includes a screen 400 of a tree structure of a design history and an input screen 402 to the tree structure.

The example of the input screen is an input screen for a box 404 of the tree structure, and indicates that ADH2 is selected as the target gene, disruption (deletion) is selected as the operation on the target gene, and the target effect (increase by 20 mM) of the target compound (ethanol) is input for the target microorganism (Saccharomyces cerevisiae: yeast). When the user clicks Submit, the input information is registered in the box 404 of the tree structure.

When the user computer 10 requests the server computer 12 to propose a candidate gene for genetic modification (S2), the server computer 12 refers to the design history database (S3) based on the request, reads the modification list (S3A), and issues a search query (S4) to the document database & search engine 16.

The document database & search engine 16 searches the database based on the search query, and outputs the search result to the server computer 12 (S5). The server computer 12 evaluates the search result, and outputs a proposal related to the candidate gene to be genetically modified to the user computer 10 based on the evaluation result (S6).

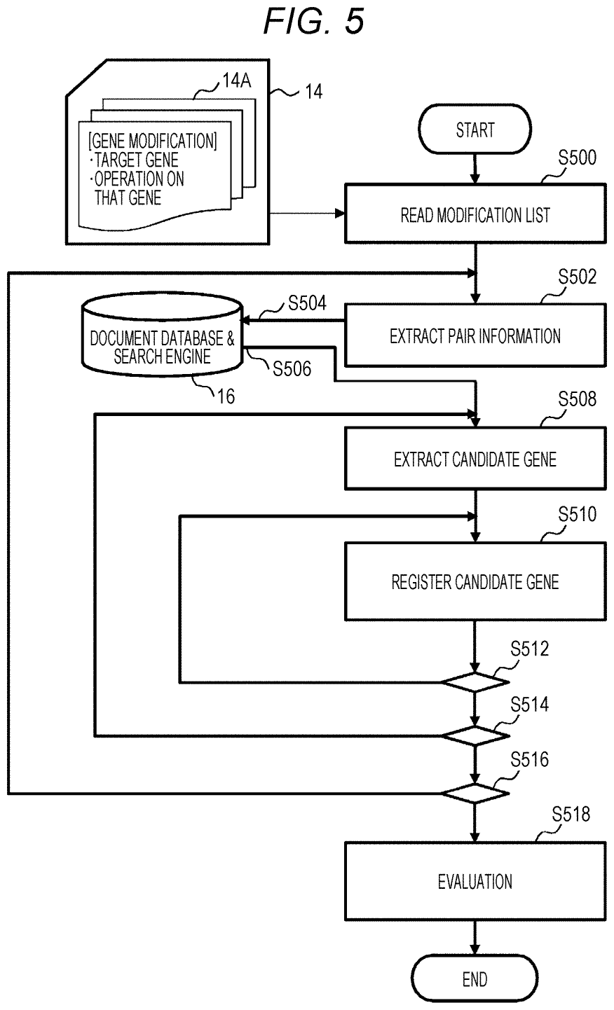

Next, the operation of the server computer 12 will be described again with reference to a flowchart (FIG. 5). The controller of the server computer 12 executes this flowchart according to a program of the memory. Upon receiving the proposal request (S2) from the user computer 10, the controller starts a flowchart.

The controller reads a modification list 14A from the design history database 14 (S500). The controller may read all the modification lists belonging to the database, or may read a modification list in a predetermined range by dividing a creation date and time of the modification list, specifying a target gene, specifying a type of operation, or the like. The user computer 10 may output the request based on an input trigger of the user, based on a predetermined number of modification lists, or based on a timed trigger such as weekly or monthly.

In S502, the controller extracts pair information of a gene name and an operation name for the gene name as search information from each of the plurality of modification lists, and sequentially outputs a search query based on the pair information to the search document database & search engine 16 (S504). The controller receives a list of related documents as a search result from the document database & search engine 16 (S506). The related document is a document in which morphemes of “target gene name” of pair information and “operation name” of the pair information exist.

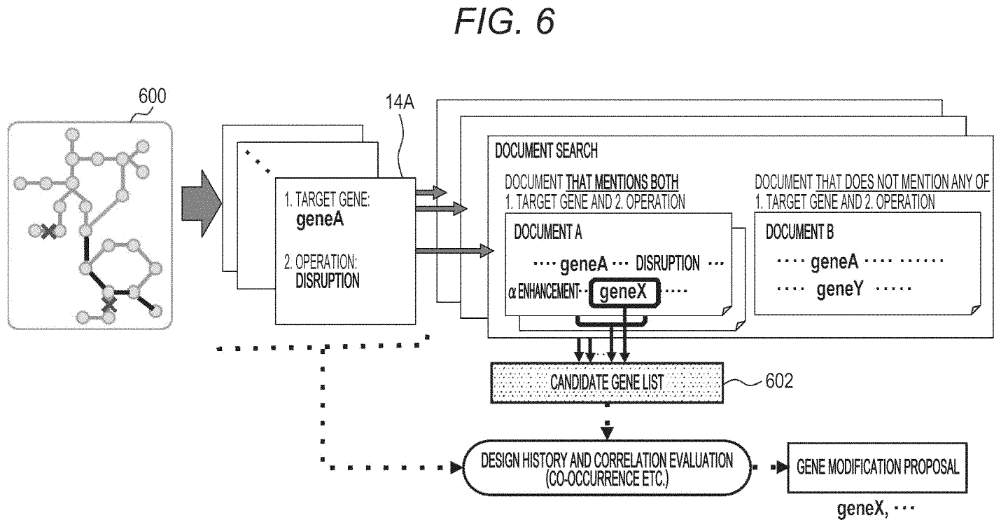

The controller processes the morphemes of the related document and extracts genes other than the target gene from the morphemes as candidate genes for improving the function of the artificial microorganism. FIG. 6 is a block diagram for explaining the principle of search of candidate genes. Reference numeral 600 denotes a metabolic map of a high-productivity microorganism.

As described above, reference numeral 14A is a modification list for the metabolic map. This modification list indicates that the target gene is “geneA” and the manipulation on the target gene is “Disruption”. This list indicates that by disrupting the “geneA”, the pathway corresponding to the “X” in the metabolic map 600 was inhibited.

The document database & search engine 16 extracts related documents including pair information (“geneA” & “Disruption”) from a plurality of documents. Documents including either “geneA” or “Disruption” or not including either of them are unrelated documents. In FIG. 6, Document A is a related document, and Document B is a non-related document.

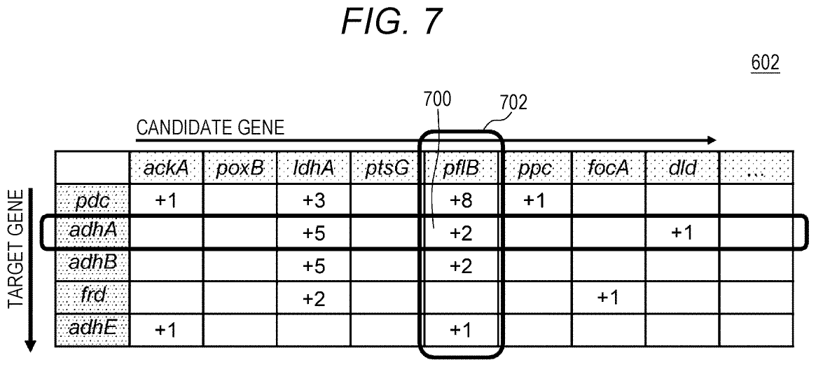

The controller analyzes morphemes of related documents, extracts genes other than the target gene (geneA) as candidate genes (S508), and registers the candidate genes in the management table 602 (S510). FIG. 7 illustrates an example of this table. The gene in the X direction is “Candidate gene”, and the gene in the Y direction is “Target gene”.

In S510, the controller registers a co-occurrence number of candidate genes in the management table 602. The “co-occurrence number of candidate genes” is the number of documents in which both the target gene and the candidate gene appear. In FIG. 7, the cell indicated by 700 indicates that the number of documents in which both adhA (target gene) and pflB (candidate gene) appear is “2”. The larger the co-occurrence number, the larger the number of documents, that is, the higher the degree of correlation between the two.

When the candidate gene does not exist in the table, the controller registers the candidate gene in the table, and registers “+1” as the co-occurrence number in the cell. When the candidate gene exists in the table, the controller adds “+1” to the co-occurrence number of the cell to update the table. A management table 602 may be recorded in the storage device of the server computer 12.

In S512, when the controller ends the processing of S508 and S510 for all the candidate genes of one related document, the controller proceeds to S514 which is the next step. When the controller ends the processing of S508 to S512 for all the related documents in S514, the controller proceeds to S516 which is the next step.

Further, in S516, when S502 to S514 are ended for all the modification lists in the design history database 14, the controller proceeds to S518 which is the next step.

The controller completes the registration of the candidate gene and the co-occurrence number of candidate genes in the management table 602 through steps S502 to S516. The co-occurrence may be referred to as frequency of occurrence.

In S518, the controller evaluates which gene among the candidate genes is excellent in modifying the properties of the biological material based on the management table 602. Therefore, the controller adds up the co-occurrence number of each candidate gene in the table 602.

Reference numeral 702 in FIG. 7 indicates that the co-occurrence number of the candidate gene (pflB) for each of the plurality of target genes is added up. The sum describes the correlation, relevance, relationship, or affinity of the candidate gene to the design history database 14. The degree of correlation indicated by reference numeral 702 is “+13”. The higher the degree of correlation, the higher the affinity, relevance, and the like of the candidate gene to the past genetic modification results as a design history database, and the higher the eligibility as a candidate gene.

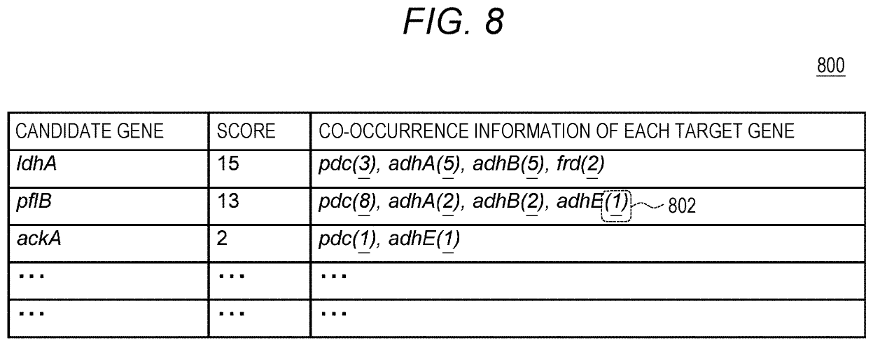

The controller evaluates the candidate genes by creating a ranking table of the candidate genes based on the overall correlation of each of all candidate genes in the management table 602. FIG. 8 is an example of a ranking table 800. The ranking table 800 has a column of correlation score and a column of co-occurrence information for each target gene for each candidate gene. The number in parentheses for each target gene is the co-occurrence number with the target gene, that is, the number of co-occurring documents. The controller may add a link to a co-occurring document to the number in parentheses.

The table 800 shows that the correlation score of the candidate gene (pflB) is “13” and the ranking is the second after the candidate gene (ldhA). Then, it indicates that, among the plurality of target genes, the co-occurrence number of “pdc” is the highest, and the correlation with “pdc” is higher than that of other target genes. The user can preferentially refer to documents that co-occur with “pdc”.

The controller outputs the ranking table 800 to the user computer 10 as a proposal related to the candidate gene (S6). The user computer 10 may notify the user of the ranking table 800, or may select and notify a candidate gene of a high ranking. The user computer 10 may select and notify a gene having a score equal to or higher than a predetermined threshold as a candidate gene.

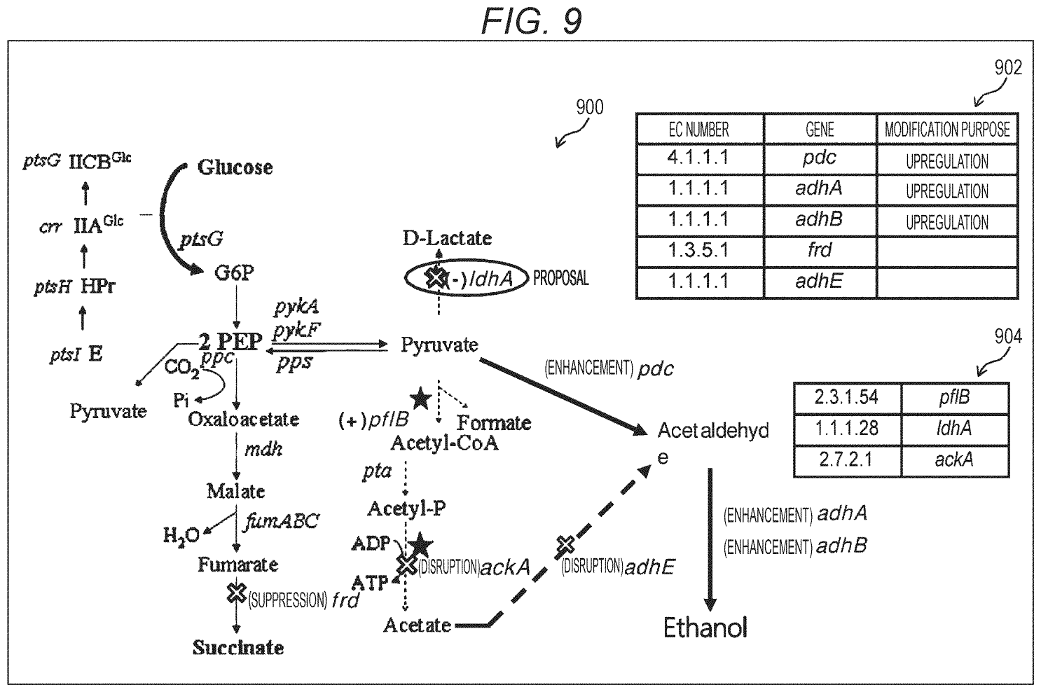

FIG. 9 is a block diagram illustrating an example of a relationship among a metabolic map 900, a target gene 902, and a candidate gene 904. The metabolic map 900 includes a metabolic pathway from glucose to ethanol production by E. coli. In the metabolic map 900, the target gene 902 and an operation on the target gene are added.

Further, in the metabolic map, disruption or suppression of “ldhA” (candidate gene: 902), which is a gene for producing an enzyme that catalyzes a metabolic pathway from pyruvate to D-lactate, and enhancement of “pflB” (candidate gene: 902), which is a gene for producing an enzyme that catalyzes a metabolic pathway from pyruvate to acetyl CoA, are added.

The server computer 12 may weight a plurality of documents belonging to the document database & search engine 16 according to a predictive coding method. A researcher or an expert flags a predetermined number of documents (teacher data) in advance whether the documents are related to improvement of a biological material. The AI mounted on the server computer 12 learns the weighting of the morphemes of each document based on the flag.

The AI scores the remaining documents in the document database & search engine 16 based on the learning result and performs ranking of the plurality of documents. The server computer 12 can instruct the document database & search engine 16 to search for documents within a predetermined ranking.

The controller can extract candidate genes from relevant documents by applying weighting to the documents, so that a candidate gene having a higher correlation can be determined with respect to the target gene.

When determining the number of co-occurrence of candidate genes, the controller adds up the number of co-occurrence with all the target genes, but may narrow down the number of co-occurrence to a predetermined target gene instead of all the target genes. This narrowing down may be selected by the user, or the target gene of which the number of times of co-occurrence is to be calculated may be narrowed down according to the existence frequency of the target gene in all the modification lists of the design history database 14.

The controller counts the number of co-occurrences in a document unit, but is not limited thereto. For example, the number of co-occurrences may be counted in a paragraph unit or a sentence unit of a related document. Then, the co-occurrence rate of the candidate gene may be calculated based on the appearance rate (the number of appearances with respect to the total number of morphemes) of each document. In addition, in the related document, a candidate gene having a small number of morphemes with respect to the target gene may be highly evaluated.

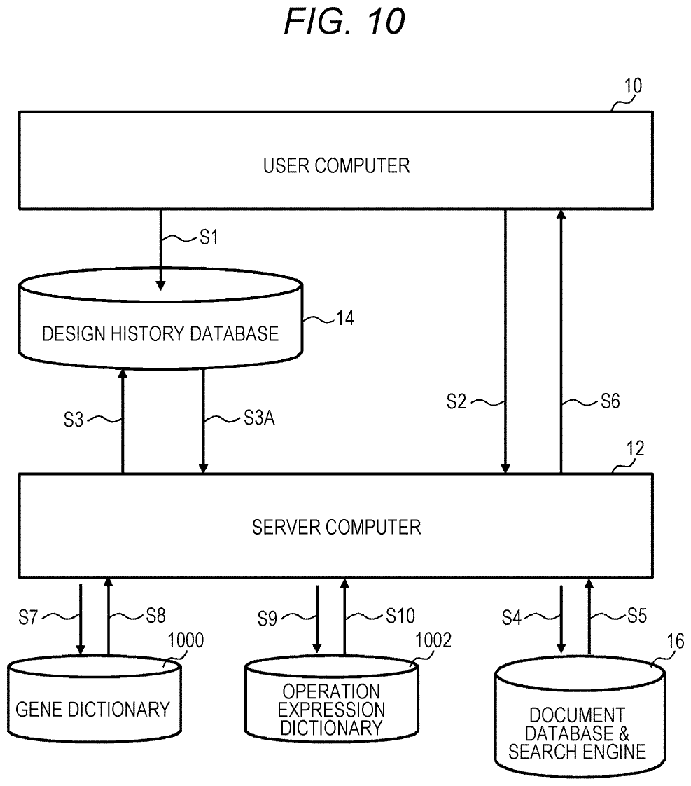

Next, a second embodiment will be described. FIG. 10 is a block diagram of the computer system. This embodiment is different from the embodiment of FIG. 1 in that a gene dictionary 1000 and an operation expression dictionary 1002 are connected to the server computer 12. In the gene dictionary 1000, genes having the same or similar functions are collected as the same enzyme number.

The operation expression dictionary 1002 collects operation names corresponding to a relationship between operation names of the modification list and synonyms or near synonyms (words having different word forms but the same or similar meanings). Near synonyms may be defined as including synonyms. Synonyms and near synonyms may be defined as related data elements. For example, morphemes having the same meaning such as “Disrupt”, “Remove”, “Delete”, and “Erase” are in a relationship of synonyms with each other.

The controller refers to the gene dictionary 1000 (S7) to acquire a gene list having the same enzyme number as the target gene of the modification list (S8), and further refers to the operation expression dictionary 1002 (S9) to acquire a list of operation names associated with operation names of the modification list (S10). The controller searches the database based on the gene list & operation list.

The controller determines a document including both at least one gene name included in the gene list and at least one operation name included in the operation list as a related document. According to the second embodiment, candidate genes can be collected in a wider range than in the first embodiment.

Next, a third embodiment will be described. This embodiment is different from the second embodiment in that a compound dictionary is further connected to the server computer 12, and a modification target compound is included in the modification list. The compound to be modified is a compound whose production amount is to be increased by an artificial microorganism by introduction, suppression, or the like of a gene.

The compound dictionary includes a compound related to the compound to be modified, for example, a precursor, a derivative, a decomposition product, or a compound having different nomenclature but the same. When acquiring the modification target compound from the modification list, the controller refers to the compound dictionary to acquire a related compound list related to the modified compound.

The controller determines a document including at least one gene name included in the gene list, at least one operation name included in the operation list, and at least one related compound included in the compound list as a related document. According to the third embodiment, it is possible to collect candidate genes more suitable for modification of artificial microorganisms in a wider range as compared with the second embodiment.

The embodiment described above is a case of the present invention, and the technical scope of the present invention is not limited to that described in the embodiment. In the embodiment described above, the technical matters described as the functions of the computer system can be specified by the terms means and elements. The function may be realized not only by software but also by hardware or a combination of hardware and software.

What is claimed is:

1. A computer system that supports design for improving a function of a biological resource, the computer system comprising a controller that executes a program recorded in a memory, wherein
the controller is configured to:
register a history of the design, wherein the history of the design includes pair information of a related element related to a property of the biological resource and an operation on the related element;
search a database based on the pair information;
acquire, based on a result of the search, additional information other than the related element, the additional information being information related to a property of the biological resource;
compute a correlation of the additional information to the related element; and
evaluate the additional information based on the calculated correlation.

2. The computer system according to claim 1, wherein
the biological resource is an artificial microorganism,
the related element is a gene, and
the additional information is a candidate gene that can be a candidate for improving the function of the artificial microorganism.

3. The computer system according to claim 2, wherein
the database includes a plurality of documents, and
the controller is configured to:
extract a related document including the pair information from the plurality of documents; and
extract the candidate gene from the related document.

4. The computer system according to claim 3, wherein
the controller performs the calculation of the correlation by counting the number of documents in which the candidate gene is described.

5. The computer system according to claim 3, wherein
the controller calculates a correlation with the candidate gene for each of a plurality of genes as the related element.

6. The computer system according to claim 5, wherein
the controller is configured to:
add up correlations of the candidate gene with each of the plurality of genes; and
evaluate a plurality of candidate genes based on the summed correlation.

7. A method for supporting design for improving a function of a biological resource by a computer, the method comprising:
by the computer,
registering a history of the design, wherein the history of the design includes pair information of a related element related to a property of the biological resource and an operation on the related element;
searching a database based on the pair information;
acquiring, based on a result of the search, additional information other than the related element, the additional information being information related to a property of the biological resource;
computing a correlation of the additional information to the related element; and
evaluating the additional information based on the calculated correlation.
